(19) United States
(12) Patent Application Publication
(10) Pub. No.: US 2021/0366583 A1
(43) Pub. Date: Nov. 25, 2021

(54) System and Method for Secure Unique Patient Identifier Generation

(71) Applicant: Merritt Seshul, Raleigh, NC (US)

(72) Inventor: Merritt Seshul, Raleigh, NC (US)

(21) Appl. No.: 16/880,031

(22) Filed: May 21, 2020

(51) Int. Cl. G16H 10/60; G16H 40/67; H04L 9/06; G06K 19/06

(52) U.S. Cl. CPC G16H 10/60; G16H 40/67; G06K 19/06037; G06K 19/06028; H04L 9/0643

(57) ABSTRACT

A system and method are presented for creating and using a Universal Patient Identifier (UPI) to ensure that records relating to a particular patient are accurately grouped only with documents relating to that patient. In an embodiment, the UPI is created by a hashing algorithm accepting patient-specific randomized and concatenated input. The first 32 bits of the resulting 512-bit hash is then used by an algorithm to perform a quick check for data discrepancies. If discrepancies are present, the system implements the much longer analysis of the entire 512-bit key to verify patient identity and record correlation.

### COPYRIGHT NOTICE

A portion of the disclosure of this patent document contains material which is subject to copyright protection. The copyright owner has no objection to the facsimile reproduction of the patent document or the patent disclosure, as it appears in the Patent and Trademark Office patent file or records, but otherwise reserves all copyright rights whatsoever.

### BACKGROUND

Cloud-based data storage solutions seek to store massive amounts of data in the most accessible manner possible, often foregoing security measures for sake of convenience, ease of use, or accessibility. Network-attached storage, local storage, and file-system-connected data storage methods are dependent on operating systems to provide users access to their files. This dependence leaves the stored data open to be compromised through misattribution of record identity or access by unauthorized actors.

The quantity increase in raw medical data per person, along with the cost benefits sought through the centralization and ready accessibility of medical data generated by different practitioners has led to a medical record consolidation rush. Such rush puts pressure on data storage systems' abilities to maintain security.

Data security and data integrity are integral to network and computer security. Even in the absence of bad actors, misattributed or mishandled medical data compromises the value of the data as well as the decisions made by professionals relying upon that data.

### BRIEF DESCRIPTION OF THE DRAWINGS

Certain illustrative embodiments illustrating organization and method of operation, together with objects and advantages may be best understood by reference to the detailed description that follows taken in conjunction with the accompanying drawings in which:

FIG. 1 is a process flow view diagram consistent with certain embodiments of the present invention.

FIG. 2 is a process flow view of a first subroutine consistent with certain embodiments of the present invention.

FIG. 3 is a process flow view of a second subroutine consistent with certain embodiments of the present invention.

### DETAILED DESCRIPTION

While this invention is susceptible of embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings.

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

Reference herein to “QR code” is to a machine-readable optical label otherwise known as a “Quick Response” code. The label typically contains information about the object to which it is attached.

Reference herein to “SQRC” is to a QR code that has data reading restrictions and is a registered trademark of DENSO WAVE Incorporated.

Reference herein to “FrameQR” is to a QR code that incorporates a flexible-use “canvas area” for custom design.

As more medical records are being digitized and stored in cloud-based servers, the need to ensure proper chain-of-identity (with regard to a particular patient) and chain-of-custody (with regard to who is authorized to access and alter medical records) is heightened as never before. This need is even more pronounced with the increasing consolidation of patient records from different and disparate systems to be merged for a particular patient.

Thus, there is a need for a system of verification to determine that medical records being accessed in cloud-based storage servers belong to a particular patient and are subject to certain safeguards as to custody.

This document discloses embodiments that relate to a universal patient record validation process. In a principal embodiment, the process validates the certainty with which all medical records relating to an individual patient are properly identified as being associated with the given individual patient. In a non-limiting example, medical records relating to an individual patient may include information relating to specific diagnostic and therapeutic medical equipment used by a patient. In an embodiment, the process need be implemented only once per patient, and need not be repeated each time the patient switches providers, has a new procedure, or when an audit occurs.

In an embodiment, the Universal Patient Identifier (UPI) of the present innovation may be used by at least medical records keepers, medical practitioners, third-party providers of medical services, surgical personnel, insurance companies, auditors, hospital record keepers, or any entity that must verify the identity of a particular patient and must ensure that all records relating to that particular patient are accurately and properly grouped only with documents relating to that particular patient.

In a principal embodiment, a system-generated numeric string directly relates to hashed demographic and/or bibliographic data, rather than a randomly generated numeric code that is untethered to the underlying data. In an embodiment, the present invention utilizes the combination of data concatenation and hashing to create unique identifiers that are directly tethered to the actual patient data. In such embodiment a user enters discrete data fields including, by way of non-limiting example, a patient's first name, the patient's last name, the patient's year of birth, and the last four digits of the patient's social security number. In a preferred embodiment, the number of data fields is limited to four, but the system may use any number of data fields. The data fields are input into any suitable randomization algorithm, such as, by way of non-limiting examples, Las Vegas algorithms, including quicksort and quickselect and Monte Carlo algorithms. The data fields are placed in a random order and the resulting randomly ordered data fields are concatenated into a single data string. This single randomized and concatenated data string is input as the seed to a hash algorithm.

In an embodiment, the UPI is created by the hashing algorithm accepting as input the randomized and concatenated data string. The hashing algorithm may be, by way of non-limiting example, the Secure Hash Algorithm SHA3-512. The algorithm may be used to hash the input data string, the first 32 bits of the resulting hash then being used by an algorithm to perform a quick-check for data discrepancies. Such data discrepancies may arise from any number of sources, including by way of non-limiting example, patient records improperly identified as belonging to a particular patient. In such embodiment, the hash itself creates a key that is 512 bits in length, a key-length that is too long to perform a quick patient identification check. However, it is reasonable to assume that the first 32 bits of the hash-created key are going to be unique most of the time. In an embodiment, the system performs a quick-check of this initial 32 bits of the hash-created key to determine the presence of any immediately-identifiable discrepancies. If the instant innovation determines that discrepancies are present, then the system will then implement the much longer analysis of the entire 512-bit key to verify patient identity and patient record correlation.

In an embodiment, this first 32 bits is the UPI, and is attached to all electronic medical records correlated to a particular individual patient. In an embodiment, the 32-bit key may be used to create a unique QR code (such as, by way of non-limiting examples, SQRC code or FrameQR) or some other unique machine-readable bar code.

In an embodiment, the system may require the check of the entire 512-bit key in certain situations regardless of the presence of discrepancies in the first 32 bits of the hash-created key. This full key check allows the system to provide an additional layer of correlation analogous to a two-step verification process.

Regardless of the embodiment, the instant innovation performs checks in two steps: an initial quick-check of the first 32 bits of the hash-created key, and an optional check of the entire 512-bit key in the case that there is a discrepancy in the quick-check or if the system determines the need for additional verification. In an embodiment legitimate patient data is limited to a pool of shared patient demographic and/or bibliographic data. Additional verification may use an alternative data element from the pool, such alternative data element purporting to identify a particular patient.

In an embodiment, the actual source of any given abnormality is irrelevant. It is enough that an abnormality exists to trigger the longer check of the entire 512-bit key. In the presence of abnormalities, the system returns a determination that the code being compared is consistent or inconsistent with the code to which it is being compared.

If the instant innovation determines the absence of abnormalities, the patient identification is verified and the patient records correlated with that identification may be accessed.

In an embodiment, the system may be implemented through various architectural configurations with a central server and archiving locations that may be co-located with the central server or remotely located through network connections to other servers and storage locations. The system may use a centralized public cloud server using public cloud storage locations. Alternatively, the system may utilize a private cloud or dedicated hardware server for the central server, and private cloud storage or local, off-cloud storage.

In an embodiment, a device associated with an end user may interact with the central server and initiate transfers to, and request transfers of digital data from, the central server. The end-user device may be implemented as a mobile device such as cell, mobile, or smartphone, a tablet form factor device, a laptop form factor device, a desktop form factor device, a network computer form factor device, or any similar end-user client device having network communication capability either through wired or wireless connections. The end-user device may also be implemented as a server form factor device.

In an embodiment of the invention, a Client may select a digital file, or files, accessible from their system on local storage device co-located with the Client, a remote storage device, or cloud storage device, and trigger the file transmission and secure storage method. This method can be initiated through a Client request. In a non-limiting example, a user logging into the web application and starting a file transfer may initiate the file transmission and secure storage method. It could also be initiated through an Application Programming Interface (API) on behalf of a user through another application.

In an embodiment, the Client instructs the system to compute a hash, or a one-way, unique representation of the input data. This hash is performed by Client directed software modules or devices which support these functions. This hash is transmitted to the central server through a secure transmission channel and connection.

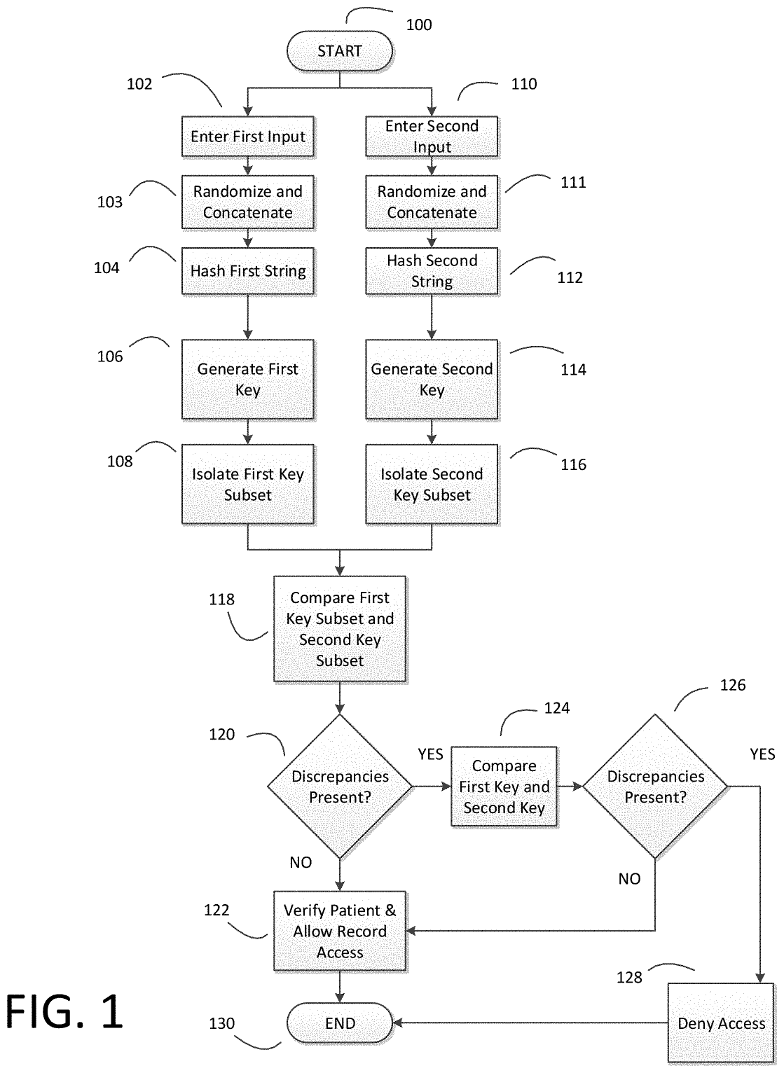

Turning now to FIG. 1, a process flow view diagram consistent with certain embodiments of the present invention is shown. At 100, the process starts. A user inputs first patient data at 102. This patient data may include, but is not necessarily limited to, a patient's first name, last name, year of birth, and the last four digits of the patient's social security number. At 103, the first input patient data is subjected to a randomization algorithm and the resulting randomized data set is concatenated to form a first string. At 104, the resultant first string is hashed using a hash algorithm such as, by way of non-limiting example, SHA3-512. This hash algorithm produces a 512-bit First Key at 106. At 108 the system isolates a First Key Subset, such First Key Subset consisting of the first 32 bits of the full 512-bit First Key. A user (who may be the same or a different individual from the user inputting the first patient data) inputs second patient data at 110. This patient data may include, but is not necessarily limited to, a patient's first name, last name, year of birth, and the last four digits of the patient's social security number. At 111, the second input patient data is subjected to a randomization algorithm and the resulting randomized data set is concatenated into a second string. At 112, the resultant second string is hashed using the same hashing algorithm used to hash the first input. At 114 the hash algorithm produces a 512-bit Second Key. At 116 the system isolates a Second Key Subset, such Second Key Subset consisting of the first 32 bots of the full 512-bit Second Key. At 118 the system compares the First Key Subset and the Second Key Subset. If at 120 the system detects no discrepancies between the two compared subsets, then at 122 the system verifies the patient and allows a user access to patient records. If at 120 the system detects discrepancies between the two compared subsets, then at 124 the system compares the First Key to the Second Key. If at 126 the system detects no discrepancies between the two compared keys, then at 122 the system verifies the patient and allows a user access to patient records. If at 126 the system detects discrepancies between the two compared keys, then at 128 the system denies access to patient records. The process ends at 130.

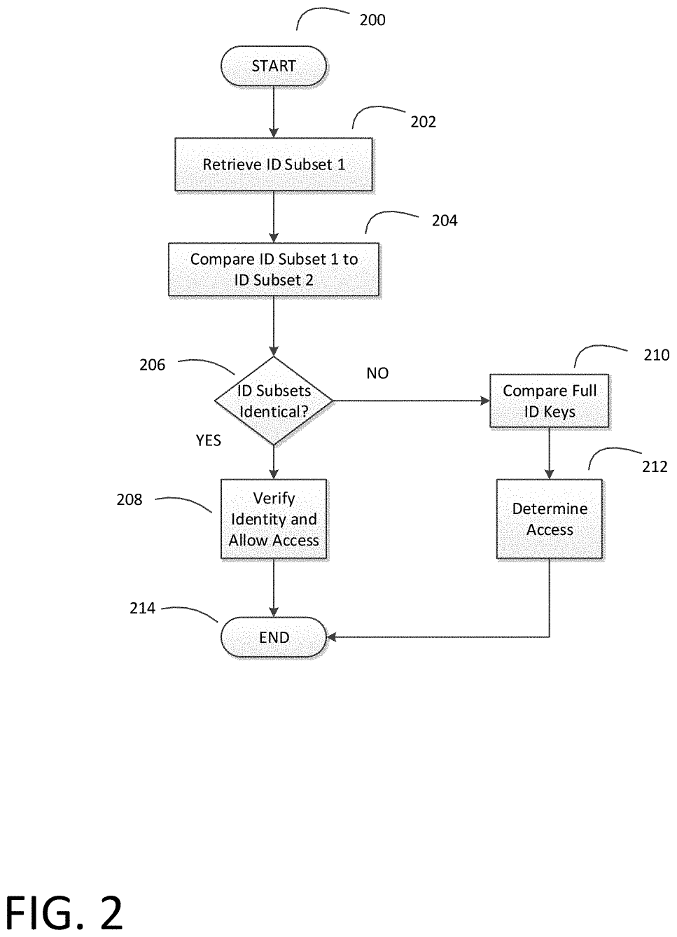

Turning now to FIG. 2, a process flow view of a first subroutine consistent with certain embodiments of the present invention is shown. At 200 the subroutine starts. At 202 the system retrieves a subset of a previously hashed identification key. In this non-limiting example, ID Subset 1 is equal to ABC, where ABC represents the information contained in the first 32 bits of a full 512-bit First Key. At 204 ID Subset 1 is compared to ID Subset 2, where ID Subset 2 consists of the first 32 bits of a full 512-bit Second Key. At 206, if ID Subset 2 is equal to ABC, then at 208 the system verifies patient identity and allows user access to patient records. If at 206 ID Subset 2 is non-identical to ID Subset 1, then the system performs a full ID Key Comparison at 210, determining a user's access grant at 212. At 214 the subroutine ends.

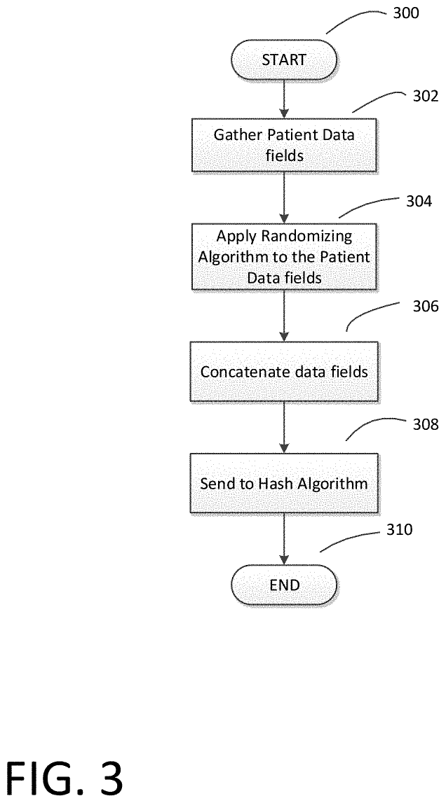

Turning now to FIG. 3, a process flow view of a second subroutine consistent with certain embodiments of the present invention is shown. At 300 the subroutine starts. At 302 the system gathers patient-specific input data such as, but not limited to, a patient's first name, the patient's last name, the patient's year of birth, and the last four digits of the person's social security number. In an embodiment the input data is represented as four discrete data fields. At 304 the data fields are input to a randomizing algorithm such as one employing, by way of non-limiting example, Las Vegas or Monte Carlo randomization. At 306 the system concatenates the randomized output, thus concatenating the patient-specific character fields in a random order to create a patient-specific character string and thereby combining the randomly ordered data fields into a single patient-specific character string without any gaps. At 308 the resulting randomized and concatenated patient-specific character string is utilized to form the seed for a hash algorithm, which then hashes the patient-specific character string through operation of the hash algorithm. At 310 the subroutine ends.

While certain illustrative embodiments have been described, it is evident that many alternatives, modifications, permutations and variations will become apparent to those skilled in the art in light of the foregoing description.

I claim:

1. A system for secure patient verification, comprising:
a server;
a processor having network connections to one or more networked storage locations;
the processor receiving a set of patient-specific character fields from a user through a network connection;
said processor concatenating said patient-specific character fields in a random order to create a patient-specific character string;
encrypting said patient-specific character string and generating an identifier for each data component associated with the patient-specific character string, said identifier capable of division into subsets;
comparing a first subset of a first identifier to a second subset of a second identifier, where both identifiers purport to identify the same patient;
verifying the association of the identifiers to the same patient; and
selectively permitting user access to each data component.

2. The system of claim 1, where the patient-specific character string is encrypted using Secure Hash Algorithm SHA3-512.

3. The system of claim 1, where the identifier for each data component associated with the patient-specific character string is a 512-bit key resulting from application of Secure Hash Algorithm SHA3-512.

4. The system of claim 1, where the first subset of a first identifier is the first 32 bits of a 512-bit key resulting from a first application of Secure Hash Algorithm SHA3-512.

5. The system of claim 1, where the second subset of a second identifier is the first 32 bits of a 512-bit key resulting from a second application of Secure Hash Algorithm SHA3-512.

6. The system of claim 1, where the first subset is converted to a machine-readable optical code and attached to all patient records.

7. The system of claim 1, where the second subset is converted to a machine-readable optical code.

8. A method for secure patient verification, comprising:
establishing network connections to one or more networked storage locations;
receiving a set of patient-specific character fields;
concatenating said patient-specific character fields in a random order to create a patient-specific character string;
encrypting said patient-specific character string and generating an identifier for each data component associated with the patient-specific character string, said identifier capable of division into subsets;
comparing a first subset of a first identifier to a second subset of a second identifier, where both identifiers purport to identify the same patient;
verifying the association of the identifiers to the same patient; and
selectively permitting user access to each data component.

9. The system of claim 8, where the patient-specific character string is encrypted using Secure Hash Algorithm SHA3-512.

10. The system of claim 8, where the identifier for each data component associated with the patient-specific character string is a 512-bit key resulting from application of Secure Hash Algorithm SHA3-512.

11. The system of claim 8, where the first subset of a first identifier is the first 32 bits of a 512-bit key resulting from a first application of Secure Hash Algorithm SHA3-512.

12. The system of claim 8, where the second subset of a second identifier is the first 32 bits of a 512-bit key resulting from a second application of Secure Hash Algorithm SHA3-512.

13. The system of claim 8, where the first subset is converted to a machine-readable optical code and attached to all patient records.

14. The system of claim 8, where the second subset is converted to a machine-readable optical code.
